(19) United States
(12) Patent Application Publication
(10) Pub. No.: US 2015/0087012 A1
(43) Pub. Date: Mar. 26, 2015

(54) METHOD FOR QUANTITATIVELY EVALUATING ANTIMICROBIAL ACTIVITY OF ANTIMICROBIAL SAMPLE USING DISC DIFFUSION METHOD AND SYSTEM FOR MEASURING ANTIMICROBIAL ACTIVITY USING THE SAME

(71) Applicant: INDUSTRY-ACADEMIC COOPERATION FOUNDATION YONSEI UNIVERSITY, Seoul (KR)

(72) Inventors: Jung Ho HWANG, Seoul (KR); Yun Haeng JOE, Seoul (KR)

(21) Appl. No.: 14/497,541

(22) Filed: Sep. 26, 2014

(30) Foreign Application Priority Data
Sep. 26, 2013 (KR) 10-2013-0114217

(51) Int. Cl. C12Q 1/18; G06F 19/10; G06T 7/00; C12Q 1/06

(52) U.S. Cl. CPC C12Q 1/18; C12Q 1/06; G06F 19/10; G06T 7/0012
USPC 435/32; 702/19; 382/128

(57) ABSTRACT

A method for quantitatively evaluating the antimicrobial activity of an antimicrobial sample using a disc diffusion method, and a measurement system using the method are provided. The method comprises: (a) measuring a maximum inhibition zone width (IZWₘₐₓ) for a first sample, in which a colony is expected not to be observed, using a disc diffusion method, and measuring a colony forming unit (CFU₀) value for the first sample using a colony counting method; (b) measuring a microbial growth inhibition zone width (IZWᵢ) and a colony forming unit (CFUᵢ) value for a second sample having any antimicrobial activity; (c) calculating α value and β value based on the results of steps (a) and (b) using equations (10) and (20); (d) measuring a microbial growth inhibition zone width (IZWₓ) for a measurement sample; and (e) calculating a quantitative antimicrobial activity value (η) using equation (30).

### BACKGROUND OF THE INVENTION

1. Field of the Invention

The preserve invention relates to a method for quantitatively evaluating the antimicrobial activity of a sample, and a system for measuring the antimicrobial activity using the method, and more particularly to a method for quantitatively evaluating the antimicrobial activity of an antimicrobial sample using a colony diffusion method and a disc diffusion method, and a system for measuring the antimicrobial activity using the method.

2. Description of the Prior Art

Generally, a colony counting method and a disc diffusion method are used to measure the antimicrobial activity of a sample. In the colony counting method, a microbial solution that reacted with a sample for a specific time period is spread on nutrient agar medium, after which microorganisms of the microbial solution are allowed to grow for a specific time period, and the grown microbial colonies are counted to determine the antimicrobial activity of the sample. In this method, quantitative measurement results can be obtained.

Meanwhile, in the disc diffusion method, a sample is placed on a nutrient agar medium having a specific amount of microorganisms spread thereon, after which the microorganisms are allowed to grow for a specific time period, and the size of a microbial growth inhibition zone that is created around the sample by the influence of the sample is measured. In this method, qualitative results for the antimicrobial activity of the sample can be obtained.

Specifically, the colony counting method is used in various research fields, because quantitative measurement results for antimicrobial activity can be obtained. However, this method has disadvantages in that repeated experiments need to be performed using repeated dilution ratios in order to obtain measurable colony forming units, and in that a process for reacting microorganisms with a sample and a process for allowing the reacted microorganisms to grow on nutrient agar medium are required. Thus, this method has disadvantages in terms of time and cost.

Meanwhile, in the disc diffusion method, a specific amount of microorganisms are spread on nutrient agar medium, and then a sample is placed thereon, and the microorganisms are allowed to grow. Thus, she reaction between the microorganisms and the sample occurs together with growth of the microorganisms. For this reason, the disc diffusion method is advantageous over the colony counting method in terms of testing time and cost. However, because quantitative results cannot be obtained by the disc diffusion method, this method is mainly used in tests for determining the minimal inhibitory concentration (MIC) of antimicrobial agents for inhibiting the growth of microorganisms.

### SUMMARY OF THE INVENTION

It is an object of the present invention to provide a method for quantitatively evaluating the antimicrobial activity of an antimicrobial sample, which can provide quantitative antimicrobial results in a more convenient and faster manner compared to the colony counting method in tests for measuring the antimicrobial activities of various samples having various antimicrobial agent concentrations, and thus overcome disadvantages in terms of testing cost and time.

To achieve the above object, in one aspect, the present invention provides a method for quantitatively evaluating the antimicrobial activity or a sample, the method comprising the steps of: (a) measuring a maximum inhibition zone width (IZWₘₐₓ) for a first sample, in which a colony is expected not to be observed, using a disc diffusion method, and measuring a colony forming unit (CFU₀) value for the first sample using a colony counting method;

(b) measuring a microbial growth inhibition zone width (IZWᵢ) for a second sample having any antimicrobial activity using the disc diffusion method, and measuring a colony forming unit (CFUᵢ) value for the second sample using the colony counting method;

(c) calculating α value and β value based on the results of steps (a) and (b) using the following equations (10) and (20);

$$\begin{matrix}α = ln [ {( C F {U}_{0} / C F {U}_{i} )}^{1 / {ρ}_{areal}} ] & ( 10 ) \\ β = ln [ {( ( I Z {W}_{max} ) / ( I Z {W}_{max} - I Z {W}_{i} ) )}^{1 / {ρ}_{areal}} ] & ( 20 )\end{matrix}$$

(d) measuring a microbial growth inhibition zone width (IZWₓ) for a measurement sample using the disc diffusion method; and

(e) calculating a quantitative antimicrobial activity value (η) using the following equation (30);

$$η = 1 - {( 1 - ( I Z {W}_{x} / I Z {W}_{max} ) )}^{α / β} , \qquad ( 30 )$$

wherein the CFU₀ value in step (a) is a colony forming unit value obtained when a sample containing no antimicrobial agent is added or not added, and ρₐᵣₑₐₗ may be the concentration ([mg/cm²] or [number/cm²]) of an antimicrobial agent contained in the second sample.

In an embodiment of the present invention, the colony counting method may comprise spreading a microbial solution, which reacted with the sample for a predetermined time, on nutrient agar medium, allowing microorganisms of the spread solution to grow for a predetermined time, and counting the grown microbial colonies to measure the antimicrobial activity of the sample.

In this case, measuring the colony forming unit value using the colony counting method may be performed using a charge-coupled device (CCD) camera or an image recognition means.

In another embodiment of the present invention, the disc diffusion method may be a method that comprises placing the sample on a nutrient agar medium having a predetermined amount of microorganisms spread thereon, and then allowing the microorganisms to grow for a predetermined time, and measuring the width (IZWₘₐₓ, IZWᵢ, or IZWₓ) of a microbial growth inhibition zone that is created around the sample by the influence of the sample.

In this ease, the inhibition zone width (IZWₘₐₓ, IZWᵢ, or IZWₓ) and one colony terming unit (CFU₀, or CFUᵢ) value may be measured using a charge-coupled device (CCD) camera or an image recognition means.

In still another embodiment of the present invention, the steps or calculating the α value, the β value and the antimicrobial activity value (η) using equations (10), (20) and (30) may be performed by a computer that performs operation processing.

In still another embodiment or the present invention, the method for quantitatively evaluating the antimicrobial activity of the sample may further comprise, after step (c), a step of storing the obtained α value, β value, CFU₀ value and IZWₘₐₓ value as a database (S135).

The present invention also provides a recording medium having stored therein a program that causes a computer to execute the quantitative antimicrobial evaluation method.

The present invention also provides a system for measuring the antimicrobial activity of a sample, which comprises the above-described recording medium.

In an embodiment of the present invention, the system for measuring the antimicrobial activity of a sample may comprise:

a recording medium;

an image recognition unit 50 configured to measure a maximum inhibition zone width (IZW) and a colony forming unit (CFU) value for the sample;

an arithmetic processing unit configured to calculate a quantitative antimicrobial activity value (η) using a quantitative antimicrobial evaluation method, stored in the recording medium, based on the maximum inhibition zone width (IZW) and colony forming unit (CFU) value measured by the image recognition unit; and

a display unit 60 configured to visually display the antimicrobial activity value (η) calculated by the arithmetic processing unit.

### BRIEF DESCRIPTION OF THE DRAWINGS

The above and other objects, features and advantages of the present invention will be more clearly understood from the following detailed description taken in conjunction with the accompanying drawing, in which:

FIG. 1 is a schematic view showing a process for performing a colony counting method;

FIG. 2 is a planar schematic view showing microbial colonies produced on a medium in a Petri dish;

FIG. 3 is a schematic view showing a process for performing a disc diffusion method;

FIG. 4 is a planar schematic view showing a clear zone created by the disc diffusion method;

FIG. 5 is a graphic diagram showing the results of performing the colony counting method and the disc diffusion method using E. coli at varying concentrations of an antimicrobial agent;

FIG. 6 is a graphic diagram showing the results of performing the colony counting method and the disc diffusion method using S. epidermidis at varying concentrations of an antimicrobial agent;

FIG. 7 is a graphic diagram showing the antibacterial efficiency (i.e., quantitative antimicrobial activity value (η)) of an antimicrobial agent as a function of the concentration thereof;

FIGS. 8 to 10 are flowcharts showing a method for quantitatively evaluating the antimicrobial activity of an antimicrobial sample according to the present invention; and

FIG. 11 is a schematic side view of a system for measuring antimicrobial activity using a quantitative antimicrobial evaluation method according to the present invention.

### DETAILED DESCRIPTION OF THE INVENTION

Hereinafter, preferred embodiments of the present invention will be described in detail with reference to the accompanying drawings, but the scope of the present invention is not limited thereto. In the following description, the detailed description of known elements will be omitted, and the detailed description of elements that may unnecessarily obscure the subject matter of the present invention will also be omitted.

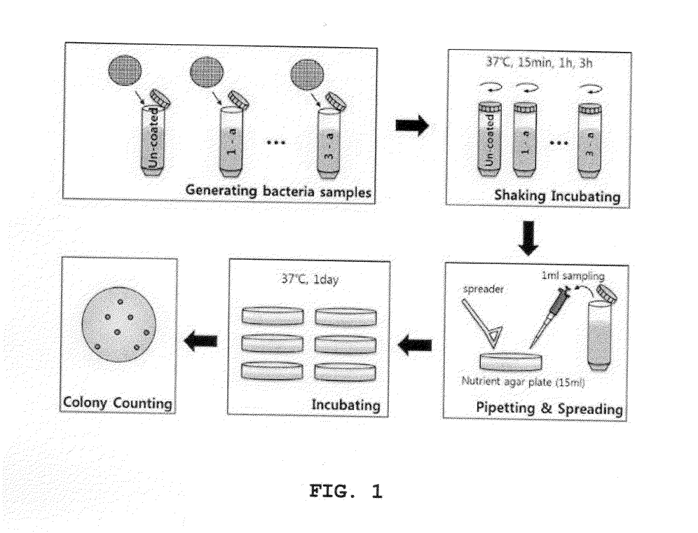

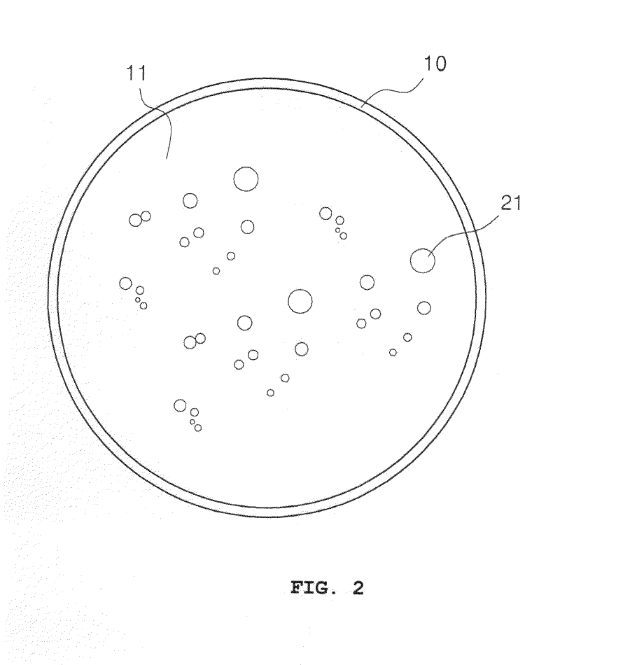

FIG. 1 is a schematic view showing a process for performing a colony counting method, and FIG. 2 is a planar schematic view showing microbial colonies produced on a medium in a Petri dish.

Referring to these figures, the colony counting method comprises spreading a microbial solution, which reacted with the sample for a predetermined time, on nutrient agar medium, and then allowing microorganisms of the microbial solution to grow for a predetermined time, and counting the grown microbial colonies to measure the antimicrobial activity of the sample. Accordingly, the colony counting method can provide quantitative measurement results for antimicrobial activity, and thus is frequently used in various research fields.

Specifically, quantitative antimicrobial activity value (η_{COM}) generally be calculated using the following equation (1):

η_{CCM}=1−CFU/CFU₀=1−CFU* (1)

In equation (1), the CFU value is the colony forming unit value of a measurement sample, the CFU₀ value is a colony forming unit value obtained when a sample containing no antimicrobial agent is added or not added, and the CFU* value is dimensionless bacterial concentration.

The present invention is based on the use of the quantitative antimicrobial activity measurement method that is the advantage of the colony counting method.

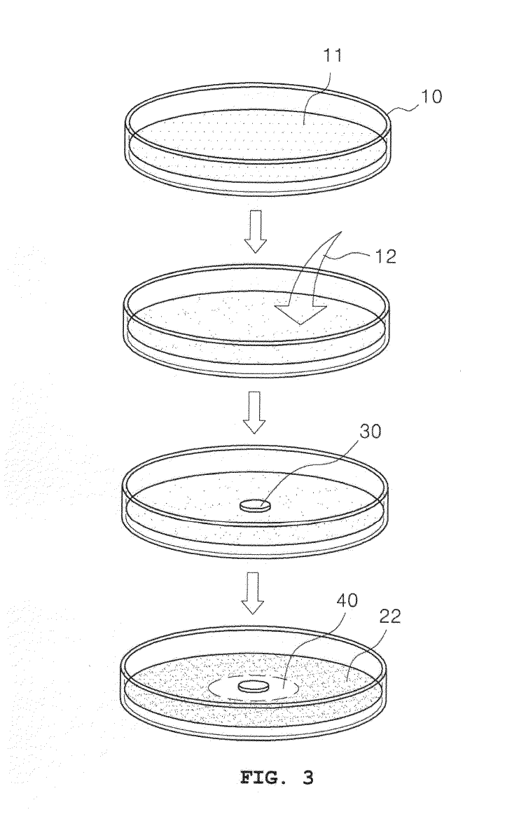

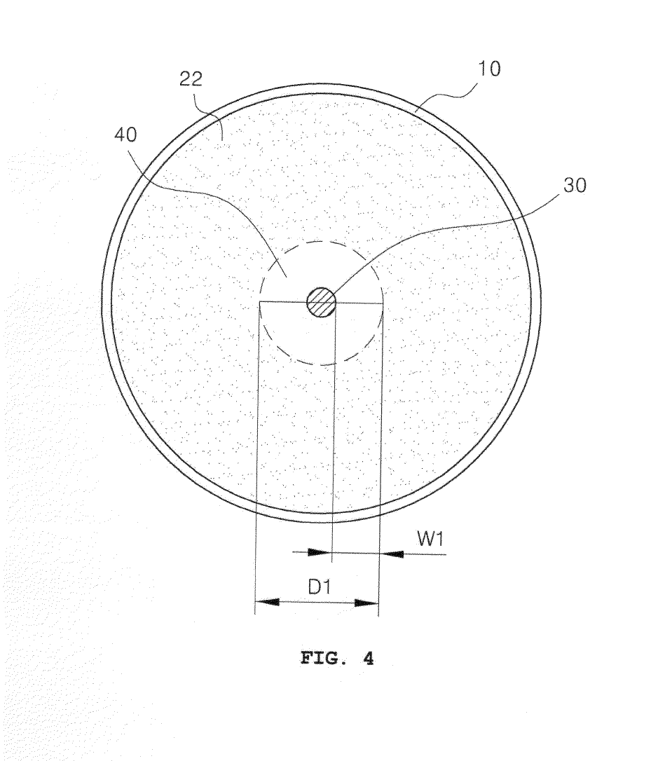

FIG. 3 is a schematic view showing a process for performing a disc diffusion method, and FIG. 4 is a planar schematic view showing a clear zone created by the disc diffusion method.

Referring these figures, in the disc diffusion method, a specific amount of microorganisms 12 are spread in a Petri dish 10 having a nutrient agar medium 11 placed therein, and a sample 30 coated with a specific amount of an antimicrobial agent is placed thereon. Then, the microorganisms are allowed to grow, and the size of a microbial growth inhibition zone (40) having a specific diameter (D1), which is created around the sample by the influence of the sample, is measured. As used herein, the phrase “size of the microbial growth inhibition zone” means the width (W1) of the zone in which microorganisms did not grow, as shown in FIG. 4.

Specifically, in the disc diffusion method, the reaction between microorganisms and a sample occurs together with growth of the microorganisms. Thus, the disc diffusion method is advantageous over the colony counting method in terms of testing time and cost. However, one disc diffusion method has a disadvantage in that it cannot provide quantitative results, unlike the colony counting method.

The present invention provides a testing method in which a quantitative antimicrobial activity measurement result that is the advantage of the colony counting method can be obtained using a convenient and cost-effective testing method that is the advantage of the disc diffusion method. This testing method will be described in detail below.

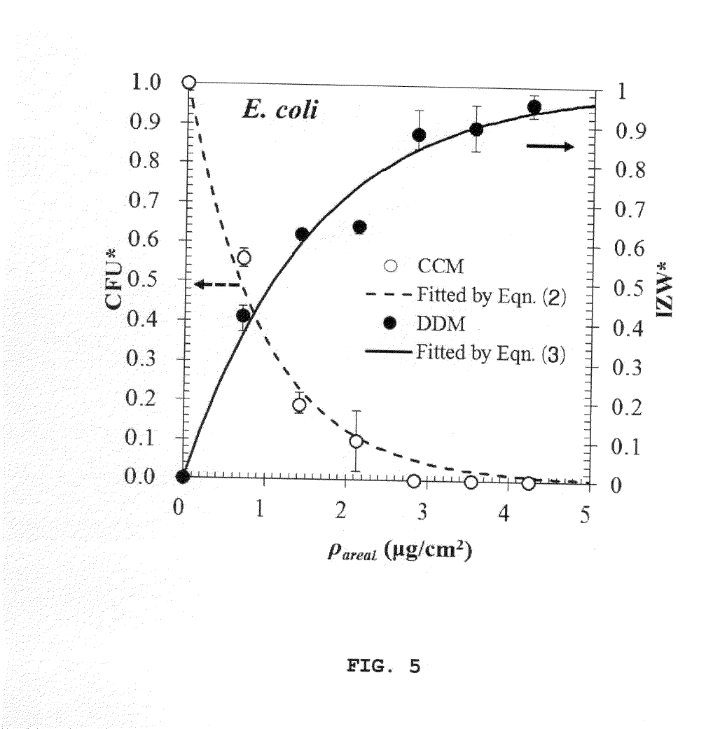

FIG. 5 is a graphic diagram showing the results of performing the colony counting method and the disc diffusion method using E. coli at varying concentrations of an antimicrobial agent.

The ρₐᵣₑₐₗ value on the X-axis of the graph is the mass per unit area of the coated antimicrobial agent, that is, the concentration of the antimicrobial agent. Herein, the unit of the concentration of the antimicrobial agent may be the mass (μg) per unit area (cm²). In some cases, the concentration may be expressed as the number per unit area (cm²). In the following description, the unit of the concentration of the antimicrobial agent is the mass (μg) per unit area (cm²).

Referring to FIG. 5, the graph (dotted line graph) showing the results of the colony counting method performed at varying concentrations (ρₐᵣₑₐₗ) of the antimicrobial agent shows that the colony forming unit (CFU*) value decreases rapidly as the concentration (ρₐᵣₑₐₗ) of the antimicrobial agent increases from 0 μg/cm² to 1 μg/cm², and then decreases slowly as the concentration (ρₐᵣₑₐₗ) of the antimicrobial agent increases toward 5 μg/cm². The dotted line graph was determined by curve fitting of the measured colony forming units and is expressed by the following equation (2).

CFU*=exp(−α×ρₐᵣₑₐₗ) (2)

In addition, the graph (solid line graph) showing the results of the disc diffusion method performed at varying concentrations (ρₐᵣₑₐₗ) of the antimicrobial agent shows that the microbial growth inhibition zone width (IZWx) increases rapidly as the concentration (ρₐᵣₑₐₗ) of the antimicrobial agent increases from 0 μg/cm² to 2 μg/cm², and then increases slowly as the concentration (ρₐᵣₑₐₗ) of the antimicrobial agent increases 5 μg/cm². The solid line graph, was determined by curve fitting of the value corresponding to the measured microbial growth inhibition zone width (IZWz) and is expressed by the following equation (3).

IZW*=1−exp(−β×ρₐᵣₑₐₗ) (3)

wherein IZW* is the dimensionless width of the microbial growth inhibition zone, defined as

IZW*=IZW/IZWₘₐₓ

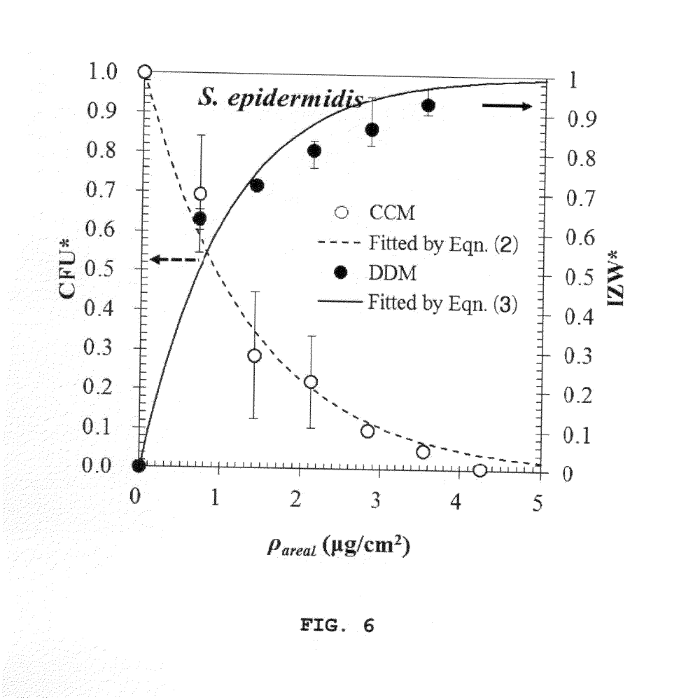

FIG. 6 is a graphic diagram showing the results of performing the colony counting method and the disc diffusion method using Staphylococcus epidermidis at varying concentrations of an antimicrobial agent.

As shown in FIGS. 5 and 6, the graphs showing the results of the colony counting method (dotted line graph) and disc diffusion method (solid line graph) performed at varying concentrations (ρₐᵣₑₐₗ) of the antimicrobial agent are substantially similar between E. coli and S. epidermidis. Thus, these graphs showing the results suggest that equation (2) and equation (3) can be applied regardless of the kind of microorganism.

When equations (2) and (3) are converted to equations that α value and β value, respectively, the following equations (4) and (5) can be obtained:

$$\begin{matrix}α = ln [ {( C F {U}_{0} / C F {U}_{i} )}^{1 / {ρ}_{areal}} ] & ( 4 ) \\ β = ln [ {( ( I Z {W}_{max} ) / ( I Z {W}_{max} - I Z {W}_{i} ) )}^{1 / {ρ}_{areal}} ] & ( 5 )\end{matrix}$$

wherein the CFUᵢ value is a colony forming unit value measured when a second sample having any antimicrobial activity is used.

Meanwhile, the following equation (6) for calculating the colony forming unit (CFU*) value can be obtained through equations (2) and (3).

CFU*=(1−IZW*)^{α/β} (6)

Thus, as can be seen in equation (6), the dimensionless colony forming unit value (CFU*) depends on the α value and the β value.

Also, using equation (6) and equation (1), the following equation 7 that indicates the quantitative antimicrobial activity value (η_{DDM}) can be obtained.

$${η}_{DDM} = 1 - {( 1 - ( I Z {W}_{x} / I Z {W}_{max} ) )}^{α / β} \qquad ( 7 )$$

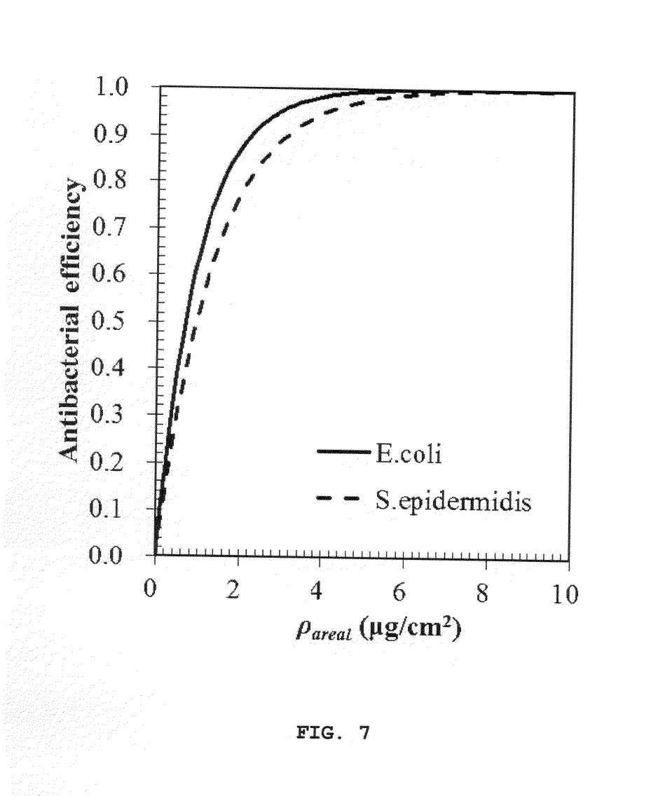

FIG. 7 is a graphic diagram showing the antibacterial efficiency (i.e., quantitative antimicrobial activity value (η)) of an antimicrobial agent as a function of the concentration thereof.

Referring to FIG. 7, the graph showing the antimicrobial activity value (η) as a function of the concentration (ρₐᵣₑₐₗ) of the antimicrobial agent is very similar between E. coli and S. epidermidis. This graph can be determined by equation (7).

The method for quantitatively evaluating the antimicrobial activity of the antimicrobial sample according to the present invention is performed using equations (4), (5) and (7). This method will be described in detail below.

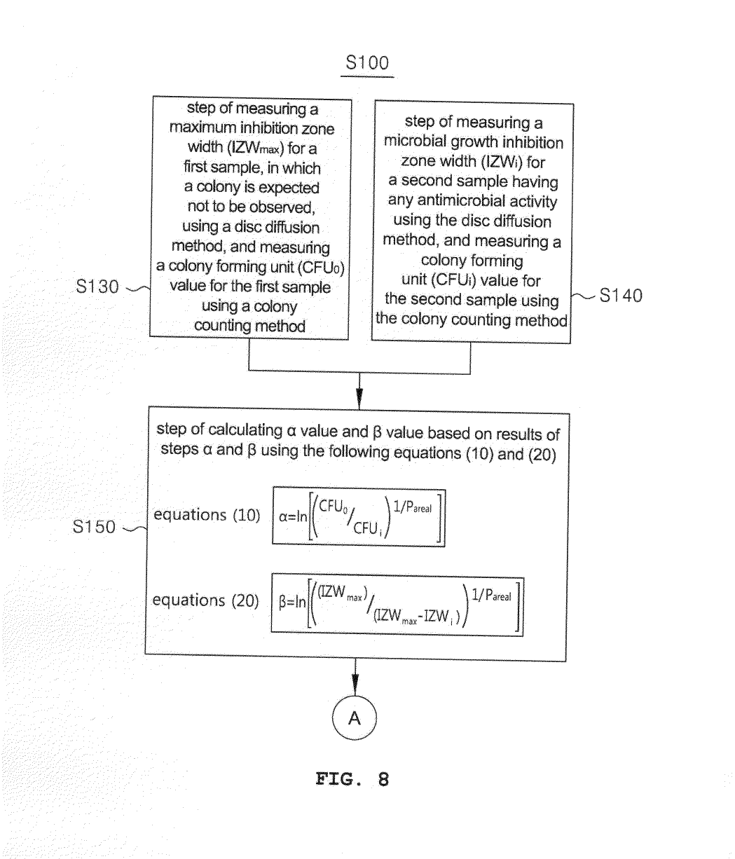

FIGS. 8 to 10 are flowcharts showing a method for quantitatively evaluating the antimicrobial activity of an antimicrobial sample according to the present invention.

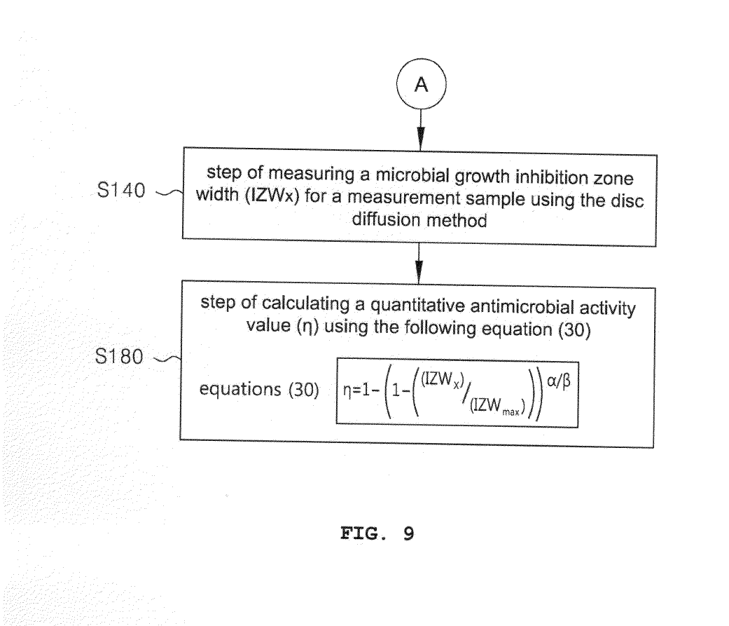

Referring to FIGS. 8 and 9, a quantitative antimicrobial evaluation method (S100) for an antimicrobial sample according to an embodiment of the present invention comprises the steps of;

(a) measuring a maximum inhibition zone width (IZWₘₐₓ) for a first sample, in which a colony is expected not to be observed, using a disc diffusion method, and measuring a colony forming unit (CFU₀) value for the first sample using a colony counting method (S110);

(b) measuring a microbial growth inhibition zone width (IZWᵢ) for a second sample having any antimicrobial activity using the disc diffusion method, and measuring a colony forming unit (CFUᵢ) value for the second sample using the colony counting method (S120);

(c) calculating α value and β value based on the results of steps (a) and (b) using the following equations (10) and (20) (S130);

$$\begin{matrix}α = ln [ {( C F {U}_{0} / C F {U}_{i} )}^{1 / {ρ}_{areal}} ] & ( 10 ) \\ β = ln [ {( ( I Z {W}_{max} ) / ( I Z {W}_{max} - I Z {W}_{i} ) )}^{1 / {ρ}_{areal}} ] & ( 20 )\end{matrix}$$

(d) measuring a microbial growth inhibition zone width (IZWₓ) for a measurement sample using the disc diffusion method (S140); and

(e) calculating a quantitative antimicrobial activity (η) using the following equation (30) (S150);

$$η = 1 - {( 1 - ( I Z {W}_{x} / I Z {W}_{max} ) )}^{α / β} , \qquad ( 30 )$$

wherein the CFU₀ value in step (a) is a colony forming unit value obtained when a sample containing no antimicrobial agent is added or not added, and ρₐᵣₑₐₗ may be the concentration ([mg/cm²] or [number/cm²]); of an antimicrobial agent contained in the second sample.

Meanwhile, although the colony forming unit value can also be visually measured, it is preferably measured by image recognition means such as a charge-coupled device (CCD) camera. In addition, although the microbial growth inhibition zone width can also be visually measured, it is measured by image recognition means such as a charge-coupled device (CCD) camera. In other words, the inhibition zone width (IZWₘₐₓ, IZW_{z}, or IZWₓ) and the colony forming unit value (CFU₀, or CFUᵢ) can be measured using a charge-coupled device (CCD) camera or an image recognition means.

The steps of calculating the α value, the β value and the antimicrobial activity value (η) using equations (10), (20) and (30) may be performed by a computer that performs operation processing.

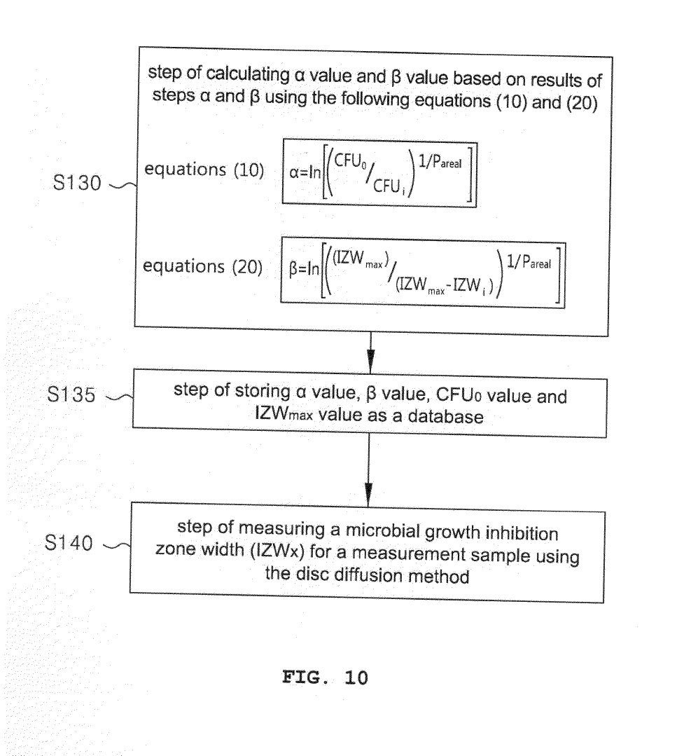

In some cases, as shown in FIG. 10, the method may further comprise, after step (c), a step of storing the obtained α value, β value, CFU₀ value and IZWₘₐₓ value as a database (S135).

Thus, for a specific type of microorganism and a specific antimicrobial agent, the α and β values in equation (30) are determined using the first sample and the second sample, after which the quantitative antimicrobial activity of a measurement sample having any antimicrobial agent concentration can be measured by performing the disc diffusion method only once.

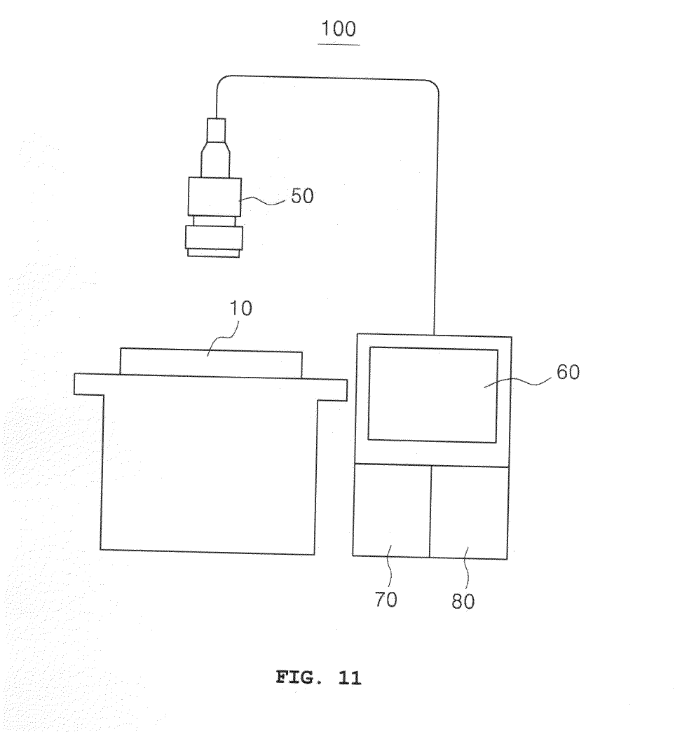

FIG. 11 shows a schematic side view of a system for measuring antimicrobial activity using a quantitative antimicrobial evaluation method according to the present invention.

Referring to FIG. 11, a system 100 for measuring the antimicrobial activity of a sample according to an embodiment of the present invention may comprise a recording medium 70, a CCD camera 50, a calculation processing unit 80, and a display unit 60.

Specifically, the recording medium 70 may have stored therein a program that causes a computer to execute the quantitative antimicrobial evaluation method of the present invention, and the CCD camera 50 may function to measure the maximum inhibition zone width (IZW) and colony forming unit (CFU) value of the sample.

Further, the calculation processing unit 60 may be configured to calculate a quantitative antimicrobial activity value (η) based on the measured values using the quantitative antimicrobial evaluation method stored in the recording medium 70, and the display unit 60 may be configured to visually display the antimicrobial activity value (η) calculated by the calculation processing unit 80.

As described above, in the quantitative antimicrobial evaluation method for the antimicrobial sample according to the present invention, a convenient and cost-effective test method that is the advantage of the disc diffusion method is applied together with the quantitative antimicrobial activity measurement method that is the advantage of the colony counting method, whereby quantitative results for antimicrobial activity can be provided in a more convenient and faster manner compared to the colony counting method in tests for measuring the antimicrobial activities of several samples having various antimicrobial agent concentrations. Thus, the quantitative antimicrobial evaluation method according to the present invention can overcome disadvantages in terms of testing cost and time.

Moreover, in the quantitative antimicrobial evaluation method for the antimicrobial sample according to the present invention, for a specific kind of microorganism and a specific antimicrobial agent, the α value, the β value, the CFU₀ value arid the CFUₘₐₓ value are stored as a database, and thus the quantitative antimicrobial activity of a measurement sample can be measured by performing the disc diffusion method only once.

In addition, the system for measuring the antimicrobial activity of a sample according to the present invention can overcome disadvantages in terms of testing cost and time by using the quantitative antimicrobial evaluation method of the present invention.

Although the preferred embodiments of the present invention have been described for illustrative purposes, those skilled in the art will appreciate that various modifications, additions and substitutions are possible, without departing from the scope and spirit of the invention as disclosed in the accompanying claims.

What is claimed is:

1. A method for quantitatively evaluating an antimicrobial activity of an antimicrobial sample, calculating a quantitative antimicrobial activity value (η) for a measurement sample by measuring a microbial growth inhibition zone width (IZWₓ) for a measurement sample using a maximum inhibition zone width (IZWₘₐₓ), a colony forming unit (CFU₀) value for a first sample, in which a colony is expected not to be observed, a microbial growth inhibition zone width (IZWᵢ), a colony forming unit (CFUᵢ) for a second sample containing a concentration ρₐᵣₑₐₗ of an antimicrobial agent and a ρₐᵣₑₐₗ.

2. The method of claim 1, wherein the method comprising the steps of:
(a) measuring a maximum inhibition zone width (IZWₘₐₓ) for a first sample, in which a colony is expected not to be observed, using a disc diffusion method, and measuring a colony forming unit (CFU₀) value for the first sample using a colony counting method;
(b) measuring a microbial growth inhibition zone width (IZWᵢ) for a second sample having any antimicrobial activity using the disc diffusion method, and measuring a colony forming unit (CFUᵢ) value for the second sample using the colony counting method;
(c) calculating α value and β value based on results of steps (a) and (b) using the following equations (10) and (20);
$$\begin{matrix}α = ln [ {( C F {U}_{0} / C F {U}_{i} )}^{1 / {ρ}_{areal}} ] & ( 10 ) \\ β = ln [ {( ( I Z {W}_{max} ) / ( I Z {W}_{max} - I Z {W}_{i} ) )}^{1 / {ρ}_{areal}} ] & ( 20 )\end{matrix}$$
(d) measuring a microbial growth inhibition zone width (IZWₓ) for a measurement sample using the disc diffusion method; and
(e) calculating a quantitative antimicrobial activity value (η) using the following equation (30);
$$η = 1 - {( 1 - ( I Z {W}_{x} / I Z {W}_{max} ) )}^{α / β} , \qquad ( 30 )$$
wherein the CFU₀ value in step (a) is a colony forming unit value obtained when a sample containing no antimicrobial agent is added or not added, and ρₐᵣₑₐₗ is a concentration ([mg/cm²] or [number/cm²]) of an antimicrobial agent contained in the second sample.

3. The method of claim 2, wherein the colony counting method comprises spreading a microbial solution, which reacted with the sample for a predetermined time, on nutrient agar medium, allowing microorganisms of the spread solution to grow for a predetermined time, and counting the grown microbial colonies to measure the antimicrobial activity of the sample.

4. The method of claim 2, wherein measuring the colony forming unit value using the colony counting method is performed using a charge-coupled device (CCD) camera or an image recognition means.

5. The method of claim 1, wherein the disc diffusion method comprises placing the sample on a nutrient agar medium having a predetermined amount of microorganisms plated thereon, and then allowing the microorganisms to grow for a predetermined time, and measuring the width (IZWₘₐₓ, IZWᵢ, or IZWₓ) of a microbial growth inhibition zone that is created around the sample by the influence of the sample.

6. The method of claim 2, wherein the inhibition zone width (IZWₘₐₓ, IZWᵢ, or IZWₓ) and the colony farcing unit (CFU₀, or CFUᵢ) value is measured using a charge-coupled device (CCD) camera or an image recognition means.

7. The method of claim 2, wherein the steps of calculating the α value, the β value and the antimicrobial activity value (η) using equations (10), (20) and (30) are performed by a computer that performs operation processing.

8. The method of claim 2, further comprising, after step (c), a step of storing the obtained α value, β value, CFU₀ value and IZWₘₐₓ value as a database.

9. A recording medium having stored therein a program that causes a computer to execute the method of claim 1.

10. A recording medium having stored therein a program that causes a computer to execute the method of claim 2.

11. A recording medium having stored therein a program that causes a computer to execute the method of claim 3.

12. A recording medium having stored therein a program that causes a computer to execute the method of claim 4.

13. A recording medium having stored therein a program that causes a computer to execute the method of claim 5.

14. A recording medium having stored therein a program that causes a computer to execute the method of claim 6.

15. A recording medium having stored therein a program that causes a computer to execute the method of claim 7.

16. A recording medium having stored therein a program that causes a computer to execute the method of claim 8.

17. A system for measuring an antimicrobial activity of a sample, the system comprising:
a recording medium;
an image recognition unit configured to measure a maximum inhibition zone width and a colony forming unit (CFU) value for the sample;
an arithmetic processing unit configured to calculate a quantitative antimicrobial activity value (η) based on the maximum inhibition zone width (IZW) and colony forming unit (CPU) value, measured by the image recognition unit, using a quantitative antimicrobial evaluation method stored in the recording medium; and
a display unit configured to visually display the antimicrobial activity value (η) calculated by the arithmetic processing unit.

18. The system of claim 17, wherein the recording medium stores therein a program that causes a computer to execute the method of claim 1.

19. The system of claim 17, wherein the recording medium stores therein a program that causes a computer to execute the method of claim 2.

20. The system, of claim 17, wherein the recording medium stores therein a program that causes a computer to execute the method of claim 3.
